(19) United States
(12) Patent Application Publication
(10) Pub. No.: US 2026/0115352 A1
(43) Pub. Date: Apr. 30, 2026

(54) Verteporfin Containing Silicone Topical Skin Adhesive for Wound Closure and Reduced Scarring

(71) Applicant: Cilag GmbH International, Zug (CH)

(72) Inventors: Duan OU, Warren, NJ (US); Mitra Vaseinasrabadi, Raritan, NJ (US); Leo B. Kriksunov, Ithaca, NY (US); Robert J. Tannhauser, Bridgewater, NJ (US); Julian A. Quintero, Flemington, NJ (US)

(21) Appl. No.: 18/928,595

(22) Filed: Oct. 28, 2024

(51) Int. Cl. A61L 26/00

(52) U.S. Cl. CPC A61L 26/0019; A61L 26/0066; A61L 2300/416

(57) ABSTRACT

The present invention is directed to topical skin adhesive formulations and kits for decreasing wound scarring comprising a rapidly curable two part liquid silicone formulation comprising a mixture of: Part A which comprises at least one vinyl terminated polydimethyl silicone base polymer, fumed silica particles, and a platinum catalyst capable of working at 20-40° C., Part B which comprises at least one vinyl terminated polydimethyl silicone base polymer and fumed silica particles, a cross-linker, a chain extender, and a medicant comprising verteporfin.

### FIELD OF THE INVENTION

The present patent invention relates to medical devices and formulations, particularly to topical skin adhesives for wound closure and healing, more particularly to silicone based rapidly curable adhesive formulations containing at least one scar-mitigating medicant, such as verteporfin.

### BACKGROUND OF THE INVENTION

Scar formation frequently occurs after surgical procedures involving incisions through one or more layers of the skin surface. Recent studies by a group of scientists in Stanford University identified a biological signal associated with scar formation and demonstrated that injecting verteporfin at the edge of the wound could mitigate the formation of the scar. Verteporfin inhibits formation of Yes-Associated Protein (YAP) which blocks the signaling of En1 activation during the wound healing process, to allow the wound repairs without fibrosis, which is attributed to scar formation. The preclinical studies performed by the Stanford university group have demonstrated regenerative skin healing in a postnatal mammal that normally scars.

PCT Publication No. WO 2023/009439A1, Mechanotransduction Disruption Mediation In Skin Grafting Methods And Compositions For Use In Practicing the Same, discloses a method of treating a wound of a subject, the method comprising: applying a skin graft to the wound in combination with a mechanotransduction blocker to treat the wound of the subject. Agents for use in combination therapy in embodiments of methods of the invention disclosed include YAP inhibitors. In some cases, the YAP inhibitor is verteporfin (benzoporphyrin derivative monoacid ring A, BPD-MA).

U.S. Patent Publication No. 2022/0313658 A1, also PCT Publication No. WO 2021/021607, YAP Inhibition for Wound Healing, discloses a method of promoting ENF-mediated healing of a wound in a dermal location of a subject, the method comprising: administering an effective amount of a YAP inhibitor composition to the wound to modulate mechanical activation of Engrailed-1 lineage-negative fibroblasts (ENFs) in the wound to promote ENF mediated healing of the wound.

PCT Publication No. WO 2023/039168 A1, Use Of Verteporfin To Modulate Wound Healing After An Ocular Surgical Procedure Or Ocular Injury, discloses a method of treating fibrosis or reducing risk of developing fibrosis or scarring in an eye of a subject after an ocular injury or ocular surgery, the method comprising administering a therapeutically effective amount of verteporfin to the subject.

U.S. Pat. No. 11,291,705, entitled Use Of Caspase-3 Inhibitors And Caspase-3 Activators In The Manufacture Of Medicament For Treating Cancer And Wound Healing, discloses a method of treating a skin wound in a subject, comprising topically administering to a wounded skin area of the subject an effective amount of a wound healing caspase-3 activator, wherein said effective amount of said caspase-3 activator is capable of increasing activity of Yes associated protein 1 (YAP) above a predetermined level as compared to a wounded area of a subject non-treated by said caspase-3 activator.

Chinese Patent Application No. CN113244163A, discloses a microneedle loaded with verteporfin for repairing scar and preparation method and application thereof, discloses a preparation method of the micro-needle for scar-carrying of verteporfin for repairing scar, wherein it comprises the following steps: S1, using laser etching silica gel substrate to obtain the microneedle template; S2, preparing hydrogel precursor solution; mixing the verteporfin in the hydrogel precursor solution, filling into the micro-needle template prepared by S1; after curing, obtaining the micro-needle.

U.S. Patent Publication No. 2016/0213757 A1, entitled Dermal Delivery, discloses a method comprising steps of: identifying a patient exhibiting at least one symptom of a disorder associated with the dermal level of the skin; and administering a nanoemulsion to the patient's skin so that at least one of the symptoms is reduced, wherein the nanoemulsion comprises a population of particles, wherein the majority of particles have diameters between approximately 10 and approximately 300 nanometers, wherein the nanoemulsion comprises: an aqueous dispersion medium; an oil; a surfactant; at least one therapeutic agent; and wherein the oil and surfactant are present at a ratio ranging between 0.5 and 2.

U.S. Patent Publication No. 2009/0005722 A1, entitled Skin-Contacting-Adhesive Free Dressing, discloses a dressing comprising an upper and lower layer, defining therebetween, a central tubular portion and spaced apart flaps, said tubular portion having an inner surface which is designed to receive a body part or plant part therein; and said upper layer having a top surface distal from said inner surface and said lower layer having a bottom surface distal to said inner surface and distal to said top surface; and securement means on the top surface of said spaced apart flaps, such that when said top surfaces of said spaced apart flaps are brought together, face to face, said securement means on the respective top surfaces of said spaced apart flaps mate with each other and secure said dressing in place without either of said spaced apart flaps being circumferentially adhered to either said central portion or to said bottom surface whereby said dressing may be applied and removed without application of substantial torque to said body part or plant part.

Chinese Patent Application No. CN111973795A, discloses a dressing for hemostasis and preventing cancer recurrence after removing liver cancer, discloses a dressing for hemostasis and anti-cancer recurrence after liver cancer resection, wherein it is prepared by the following steps: 1) preparation of verteporfin slow-release nano-micelle using glycolide and/or lactide as monomer for polymerization reaction to prepare polymer, taking the polymer as shell material, using verteporfin as raw material, taking polyethylene glycol derivatization phosphatidyl ethanolamine (PEG-PE) as carrier, using film dispersion-hydration method to prepare verteporfin slow release nano-micelle; 2) preparing the verteporfin slow-release nanometer micelle modified electrostatic spinning dressing by using core liquid containing verteporfin nanometer micelle and shell liquid for coaxial electrostatic spinning to obtain the verteporfin slow-release nanometer micelle modified electrostatic spinning dressing.

U.S. Patent Publication No. 2021/0369639 A1, entitled Novel Antimicrobial Topical Skin Closure Compositions and Systems, discloses a composition comprising: a cross-linkable silicone polymer having reactive functionalities; a silica-containing composition; a silicone cross-linking agent; a catalyst, wherein said catalyst comprises a platinum tetramethyldivinyl disiloxane diethyl maleate complex.

U.S. Patent Publication No. 2014/0303541 A1, entitled Medical Devices, Wound Dressings, and Methods for Dressing Wounds, discloses a wound dressing comprising: a substrate; and an adhesive mixture bound to the substrate, the adhesive mixture comprising tacky silicone material and at least one active pharmaceutical agent.

An article titled “Biodegradable Hydrogels With Photodynamic Antibacterial Activity Promote Wound Healing And Mitigate Scar Formation”, by Chen Zhang, et al., Biomater. Sci., 2023,11, 288-297, discloses construction of a verteporfin-loaded biodegradable hydrogel (verteporfin-gel) using hyaluronic acid and thiol-terminated 4-arm polyethylene glycol (PEG). The injectable verteporfin-gel sustainably releases small doses of verteporfin in the wound microenvironment that generates reactive oxygen species (ROS) under red light irradiation to kill bacteria efficiently.

An article “Preventing Engrailed-1 Activation In Fibroblasts Yields Wound Regeneration Without Scarring”, Mascharak et al., Science 372, 362 (2021), discloses, YAP and En1 as possible molecular targets to prevent scarring. Inhibition of YAP signaling prevents En1 activation during wound healing, thus encouraging ENF-mediated wound repair without fibrosis and with regeneration of secondary skin elements (hair follicles, sebaceous glands).

An article “To Scar or Not to Scar”, Richard Clark, The New England Journal of Medicine, 385(5),469-471, 2021, discloses molecular, cellular, and pathobiologic processes of the YAP pathway in skin-wound scarring and the ability of verteporfin to block such processes and to promote regenerative skin healing.

An article “Identification And Isolation Of A Dermal Lineage With Intrinsic Fibrogenic Potential”, Yuval Rinkevich, et al., Science, 2015 Apr. 17; 348(6232): aaa2151, discloses that dermal fibroblasts represent a heterogeneous population of cells with diverse features that remain largely undefined. It is revealed the presence of at least two fibroblast lineages in murine dorsal skin. Lineage tracing and transplantation assays demonstrate that a single fibroblast lineage is responsible for the bulk of connective tissue deposition during embryonic development, cutaneous wound healing, radiation fibrosis, and cancer stroma formation. Lineage-specific cell ablation leads to diminished connective tissue deposition in wounds and reduces melanoma growth. Using flow cytometry, CD26/DPP4 identified as a surface marker that allows isolation of this lineage. Small molecule-based inhibition of CD26/DPP4 enzymatic activity during wound healing results in diminished cutaneous scarring. Identification and isolation of these lineages hold promise for translational medicine aimed at in vivo modulation of fibrogenic behavior.

An article “Converting Fibroblastic Fates Leads To Wound Healing Without Scar”, Dongsheng Jiang and Yuval Rinkevich, Signal Transduction and Targeted Therapy (2021) 6:332, discloses treatment of YAP inhibitor verteporfin in wounds of non-inducible Cre lines (En1Cre;R26mTmG) resulted in substantial reduction of En1 lineage-positive fibroblasts (EPFs), including eEPFs that were ˜60% in wounds.

An article “Scarless Wound Healing Programmed by Core-Shell Microneedles”, Ying Zhangu et al., Nature Communications, 2023: 14:3431, discloses effective reprogramming of chronic wound healing remains challenging due to the limited drug delivery efficacy hindered by physiological barriers, as well as the inappropriate dosing timing in distinct healing stages. A core-shell structured microneedle array patch with programmed functions is designed to dynamically modulate the wound immune microenvironment according to the varied healing phases. The released verteporfin inhibits scar formation by blocking Engrailed-1 (En1) activation in fibroblasts.

U.S. Patent Publication No. 2021/0371596 A1, entitled Novel Antimicrobial Topical Skin Closure Compositions and Systems, discloses a composition comprising: a cross-linkable silicone polymer having reactive functionalities; a silica-containing composition; a silicone cross-linking agent; a catalyst, wherein said catalyst comprises a platinum tetramethyldivinyl disiloxane diethyl maleate complex, and an antimicrobial agent.

U.S. Patent Publication No. 2021/0369639 A1, entitled Novel Antimicrobial Topical Skin Closure Compositions and Systems, discloses a composition comprising: a cross-linkable silicone polymer having reactive functionalities; a silica-containing composition; a silicone cross-linking agent; a catalyst, wherein said catalyst comprises a platinum tetramethyldivinyl disiloxane diethyl maleate; and an antimicrobial agent.

U.S. Patent Publication No. 2021/0369258 A1, entitled Systems, Devices And Methods For Dispensing And Curing Silicone Based Topical Skin Adhesives, discloses a system for dispensing a curable composition comprising: a delivery device; a static mixer having a proximal end that is connected with a distal end of said delivery device; a flexible spreader having a proximal end that is connected with a distal end of said static mixer, a distal end including a flat dispensing opening, and a plurality of channels extending through said flexible spreader to said flat dispensing opening.

### SUMMARY OF THE INVENTION

In one aspect, this invention relates to a topical skin adhesive comprising a rapidly curable two part liquid silicone formulation comprising a mixture of a Part A component which comprises a vinyl terminated polydimethyl silicone base polymer, fumed silica particles, and a platinum catalyst capable of working at 20-40° C., a Part B component which comprises the vinyl terminated polydimethyl silicone base polymer and fumed silica particles, a cross-linker, a chain extender, and a medicant comprising Verteporfin (VP).

In another aspect, the medicant concentration is from 1% to 9%.

In yet another aspect, the inventive formulation is tack-free cured in under 3 minutes after mixing when applied to a skin of a mammal and forms a solid film dressing, wherein said dressing exhibits a sustained release of verteporfin.

In further aspect, this invention relates to a system for applying the topical skin adhesive, comprising: a dual barrel syringe containing in a first syringe the Part A and in a second syringe the Part B, the dual barrel syringe terminating in a static mixer configured for mixing and co-expressing the Part A and the Part B.

### BRIEF DESCRIPTION OF THE DRAWINGS

FIG. 1 shows images of cured silicone films.

FIG. 2 shows on the left a vial containing the extraction solvent used in the test—ethyl acetate; on the right is shown the vial after extraction from the verteporfin-containing silicone film, containing deep green extract.

FIG. 3 shows 1H NMR of the green residue from solvent extraction of silicone film containing verteporfin vs. control sample just containing verteporfin.

FIG. 4 shows the structure of verteporfin.

FIG. 5 shows UV-Vis absorption measurements for blank bovine plasma sample (BP sample) and bovine plasma exposed to silicone containing verteporfin (BP-verteporfin sample).

FIG. 6 presents the absorption spectra of five different verteporfin dilutions (verteporfin-IPA) and IPA blank.

FIG. 7 presents a calibration curve obtained based on Beer-Lambert law from the data of Table 3, measured at λ max i.e. at 690 nm.

FIG. 8 shows the UV-Vis absorption spectra of Bovine Plasma after incubation with silicone TSA films having 3% verteporfin, after 6 hours to one week incubation, with the lowest curve corresponding to blank sample (no verteporfin loading), and the highest curve corresponding to 1 week incubation.

FIG. 9 shows the UV-Vis absorption spectra of Bovine Plasma after incubation with silicone TSA films having 6% verteporfin, after 6 hours to one week incubation, with the lowest curve corresponding to blank sample (no verteporfin loading), and the highest curve corresponding to 1 week incubation.

FIG. 10 shows cumulative verteporfin mass release vs. time for 3% verteporfin containing silicone TSA films in Bovine Plasma.

FIG. 11 shows cumulative verteporfin mass release vs. time for 6% verteporfin containing silicone TSA films in Bovine Plasma.

### DETAILED DESCRIPTION OF THE PREFERRED EMBODIMENTS

The present invention relates generally to rapidly curable silicone based compositions containing releasable anti-scarring medicant, such as verteporfin, for application to wounds, for decreasing and mitigating scar formation. It further relates to methods of closing wounds and the delivery of verteporfin to the wound immediately after closing and during first several hours and days of wound healing. Verteporfin is contained in a liquid, rapidly curable silicone based topical skin adhesive (TSA) and is continuously released from the cured TSA film onto and into the healing tissue of the wound. As a result of the present work, no injection into and through inner layers of skin tissue is required, as the verteporfin will be absorbed through injured, ruptured or open tissue and skin that is undergoing healing and reconstruction.

Rapidly curable silicone composition refers to a liquid skin-adhesive formulation that is curable into a film on the skin of a live mammal, at ambient air temperature, such as 20-25 C, within less than 5 minutes, more preferably less than 3 minutes, such as in 2 minutes, to a tack-free film.

The inventors have surprisingly discovered that many components are not able to be homogeneously dispersed in and then released from the silicone matrix. Surprisingly, the compatibility between verteporfin and curable liquid silicone composition is good, which makes it possible to disperse it into a silicone matrix and then continuously released onto a wound.

Surprisingly, cured silicone film can act as a depot of verteporfin from which that agent can then be released from the silicone to contact the fibroblast cells on the surface of the wound. Along with this discovery was that the incorporation of verteporfin does not compromise the sealing and adhesion properties of the silicone adhesive or the rate of cross-linking/curing.

Verteporfin delivery from cured silicone films is simple, uniform, and able to be simultaneously released over an extended time over a large area without the associated challenges when using injections (e.g., difficulty in targeting and concentration control), and potential for infective pathogen ingress). Further, silicone TSA also simultaneously provides a barrier for bacterial infections. Advantageously, the silicone matrix releases verteporfin into the injured wound/skin continuously after the closure of incision with liquid silicone TSA.

An extraction study using an ester solvent on verteporfin containing silicone topical skin adhesive film indicates the verteporfin molecules readily leach out from the silicone carrier at ambient conditions. We expect that verteporfin reacts directly onto papillary fibroblasts of the freshly formed wound immediately after incision closure and penetrates deeper dermis and to react with reticular fibroblasts through the wound over time without the need in any injection, especially at the early stage of wound closure.

In some embodiments, a secondary medicant and/or Active Pharmaceutical Ingredient (API) can be further incorporated into the TSA. In some embodiments, both verteporfin and an antibacterial agent, such as triclosan, are incorporated into the rapidly curable liquid silicone TSA composition and released continuously. In some embodiments, verteporfin is incorporated into the curable liquid silicone, while the secondary medicant and/or API is incorporated into a porous mesh applied onto the skin prior to application of TSA.

According to one embodiment of the present invention, there is provided a porous patch or mesh configured for application onto a wound to approximate the wound sides and to hold the wound sides in apposition, with a verteporfin containing liquid silicone adhesive applied over the porous patch or mesh to secure the patch onto the skin over the wound to protect the wound from infection and deliver the verteporfin and/or medicant into the wound over time to improve healing.

Preferred components of a two-part curable composition are presented below.
Part A comprising
   A1) At least one vinyl terminated polydimethyl silicone base polymer component having a number average molecular weight, such as 10000-200000, more preferably a blend of a low viscosity vinyl terminated polydimethyl silicone based polymer and a higher viscosity vinyl terminated polydimethyl silicone based polymer, wherein the number average molecular weight of the low viscosity vinyl terminated polydimethylsiloxane polymer ranges from 1000 to 10000 and the higher viscosity vinyl terminated polydimethylsiloxane polymer having a number average molecular weight of 25000 to 150000;
   A2) fumed surface treated silica particles;
   A3) a platinum catalyst capable of working at 20-40° C.; such as platinum tetramethyldivinyl disiloxane diethyl maleate complex.
Part B comprising
   B1) at least one vinyl terminated polydimethyl silicone base polymer component having a number average molecular weight in the range of 25000 to 150000,
   B2) fumed surface treated silica particles,
   B3) a cross-linker that reacts with a plurality, such as three or more, available groups on the silicone base polymers, such as a polymethylhydro-co-polydimethyl siloxane cross linker,
   B4) a chain extender that can react with not more than two available groups on the silicone based polymer, such as a Si—H terminated polydimethylsiloxane chain extender, and
   B5) Verteporfin.

Preferred curable siloxane materials are Polydimethylsiloxane (PDMS) that produce cured films and articles having flexible, rubber-like properties. Exemplary curable PDMS materials, include Elkem Silbione 4020-55, which has a number average molecular weight in the range of 25000 to 150000, while the low viscosity PDMS material can be provided as a carrier for the platinum catalyst. As defined herein, unless stated otherwise, number average molecular weight can be determined based on the relationship between kinematic viscosity and molecular weight (page 11, SILICONE FLUIDS: STABLE, INERT MEDIA ENGINEERING AND DESIGN PROPERTIES, Catalog published by Gelest, Inc. 11 East Steel Rd. Morrisville, Pa. 19067). Using A. J. Barry's relationship for molecular weights (M)>2,500 correlating the kinematic viscosity expressed in centistokes (cSt) at 25° C., the molecular weight M of silicones can be estimated as follows:

$$log {μ}_{cSt} = 1. + 0.0 \; 123 {M}^{0.5} , ( as published by A . J . Barry in the Journal of Applied Physics 17 , 1020 ( 1946 ) ) .$$

The fumed silica component is also known as pyrogenic silica, and is a white, fluffy powder made from burning silicon tetrachloride (SiCl₄) in a hydrogen-oxygen flame. Fumed silica generally consists of very small particles of silicon dioxide (SiO₂). The fumed silica particles can be treated with hydrophobic surface modifications (such as with silanes) to improve compatibility with siloxanes and to prevent moisture absorption, which can affect the curing process. Hydrophobically treated fumed silica tends to disperse more easily in siloxane formulations.

The preferred siloxane crosslinking agent is methylsiloxane-dimethylsiloxane copolymer, a type of silicone hydride functional polymer that contains both methyl groups (—CH₃) and silicon-hydride (Si—H) functional groups. The presence of these functional groups allows the polymer to participate in hydrosilylation reactions (a key type of chemical reaction in silicone chemistry). The Si—H functional groups make the compounds reactive and crosslinkable with other polymers, particularly in the presence of catalysts such as platinum. Gelest HMS H301 can react with unsaturated polymers, such as vinyl silicones, to form a cured, stable silicone network and, due to its reactive hydride groups, can be used in formulations for adhesives or sealants that cure via hydrosilylation. Hydrosilylation is a process where the hydride (Si—H) groups in the polymer react with vinyl-functional silicones in the presence of a catalyst like platinum. This reaction forms a stable, cured elastomer.

The chain extender component is characterized by being able react with not more than two available groups on the silicone based polymer, such as a Si—H terminated polydimethylsiloxane chain extender. A SiH terminated polydimethylsiloxane is added as a chain extender to polymerize the low molecular weight vinyl terminated polydimentylsiloxane. The SiH terminated polydimethylsiloxane base polymer has molecular weight between 1000 and 100,000, preferably between 3,000 to 10,000. It is a silicone polymer with hydride (Si—H) groups at the ends of the polymer chains. The preferred chain extender is Gelest DMS H21, a hydride-terminated polydimethylsiloxane (PDMS).

A delivery device can be any dual barrel syringe configured for storing and expressing on demand Part A and Part B components, with a static mixer tip for expressing parts A and B as a well-mixed composition in a 1:1 ratio or alternatively over a range of ratios such as from 0.5:1 through 1:0.5 ratios.

The porous patch or mesh substrate could be a porous flat, flexible polymeric mesh, woven or non-woven, such as perforated film or fabric, having porosity from about 10% to about 95%, and having on a tissue facing side an optional biocompatible pressure sensitive adhesive. The mesh material can be any biocompatible polymer such as polypropylene, polyethylene, PET or similar, and combinations thereof.

The present silicone based rapidly curable adhesive is a platinum catalyzed two-part silicone rubber elastomer. The formulation is cured by cross linking of vinyl terminated polydimethylsiloxane in the presence of a platinum catalyst. Platinum-cured silicone rubbers often encounter the phenomenon of not curing rapidly or poor adhesion to the contact surface or not curing tacky-free. The typical culprit is that the catalytic activity of the catalyst declines or is lost due to the action of certain substances that result in catalyst poisoning. Catalytic poisons are usually impurities carried in the raw materials, or some impurities of the catalyst itself, in addition the products or by-products produced by the reaction, all of which may poison, i.e. inactivate the catalyst.

Poisoning of the catalysts is essentially due to small amounts of impurities contained in the raw materials that are either strongly adsorbed on the active center (mostly chemical adsorption), or chemically interact with the active center and become other substances, thereby poisoning the catalyst active center. The severity of “poisoning” is determined by the type and quantity of impurities. Amine compounds are the common substances that can easily cause platinum catalyst poisoning: neutralizing amine, ethanolamine, N-methyl-ethanolamine, triethanolamine, N-dimethylethanolamine, n-butylamine, diethylamine, triethylamine, tetramethylene ethylenediamine (butyl Diamine), cyclohexylamine, melamine, dimethylformamide. NH is the chemical function present in all amines which is responsible to the poisoning of platinum catalyst.

Verteporfin, a benzoporphyrin derivative, is a medication used as a photosensitizer for photodynamic therapy to eliminate the abnormal blood vessels in the eye associated with conditions such as the wet form of macular degeneration. FIG. 4 shows the structure of verteporfin.

As illustrated in the FIG. 4, two amine functions present in each verteporfin molecule. However, surprisingly and unexpectedly, a moderate dose of verteporfin loading in the instant silicone TSA was found to not diminish the reactivity of platinum catalyst used in the embodiments of the present invention and demonstrated below.

### Example 1. Preparation of Silicone TSA Containing Verteporfin

The following steps illustrate the formation of a prototype device and in-vitro testing.

### Step 1: Catalyst Preparation

2.7 grams of diethyl maleate was mixed with 3.6 grams of diethyl ether and 3.6 grams of Gelest SIP 6830.3 (3.0% platinum divinyl tetramethyldisiloxane complex in vinyl terminated polydimethylsiloxane, Karstedt catalyst xylene solvent free) at ambient temperature for 24 hours. 64.9 grams of Gelest SIP 6830.3 was then added into the above mixture and mixed for an additional 72 hours while the lid of the container remained open. Finally, 928.8 grams of vinyl terminated polydimethylsiloxane (Gelest DMS V21) was added and mixed for an additional 4 hours. The platinum catalyst master batch contains the catalyst having 2055 ppm of elemental platinum with essentially the remaining being vinyl terminated polydimethylsiloxane. The catalyst is platinum tetramethyldivinyl disiloxane diethyl maleate complex.

### Step 2. Two-Part Medicant/API Containing Silicone Adhesive Preparation.

Part A: 90 grams of Elkem 55 experimental base (also known as Elkem Silbione 4020-55, containing vinyl terminated polydimethyl silicone base polymer and fumed silica particles) was mixed with 10 grams of the platinum catalyst master batch prepared according to step 1, using a high-speed centrifugal mixer (FlackTek DAC150 FV-K) at 3470 rpm for 3 minutes. This composition had viscosity of 32,090 CPs.

Part B: 84.6 grams of Elkem 55 experimental base was mixed with 8.46 grams of polymethylhydro-co-polydimethyl siloxane cross linker (Gelest HMS H301), 2.82 grams of SiH terminated polydimethylsiloxane chain extender (Gelest DMS H21) and 6.12 grams of Verteporfin (Sigma Aldrich, SKU1711461) (corresponding to 3% verteporfin loading, with other examples substitution for 1, 3, 6, 9% verteporfin loading as shown below) using a high-speed centrifugal mixer (Flack Tek DAC150 FV-K) at 3470 rpm for 3 minutes. This composition had a viscosity of 31,160 CPs.

### Step 3. Testing Sample of Silicone Film Preparation.

Equal amount of the two—part silicone TSA (Parts A, B) composition prepared according to step 2 were mixed and applied onto a polyethylene release film using a conventional rubber spatula. The API-containing silicone film was peeled off from the polyethylene substrate after 1 hour for further testing.

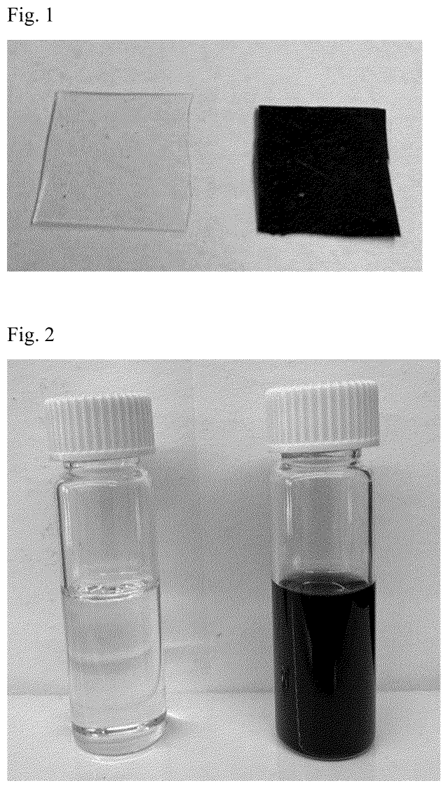

FIG. 1 shows images of cured silicone film, prepared as described above. The sample containing 3% of verteporfin homogeneously dispersed into the silicone matrix (preparation described above) is shown on the right, the dark blue color shows verteporfin evenly distributed across the entire matrix and turned the transparent silicone film into dark blue color. On the left is the same film prepared as described above but without Verteporfin.

### Step 4. Extraction Testing for the Evaluation of the Release of API (Verteporfin) in a Model Solvent.

0.5 grams of verteporfin-containing silicone film prepared according to step 3 were mixed with 5.5 grams of ethyl acetate using a Vortex mixer at ambient temperature for 1 hour. The color of the solvent turned from transparent to deep green as illustrated in FIG. 2. FIG. 2 shows on the left a vial containing the extraction solvent used in the test—ethyl acetate; on the right is shown the vial after extraction from the verteporfin-containing silicone film obtained in step 3 above, containing deep green extraction solution.

### Example 2. Characterization of the Solvent Extract from the Silicone Matrix Containing Verteporfin

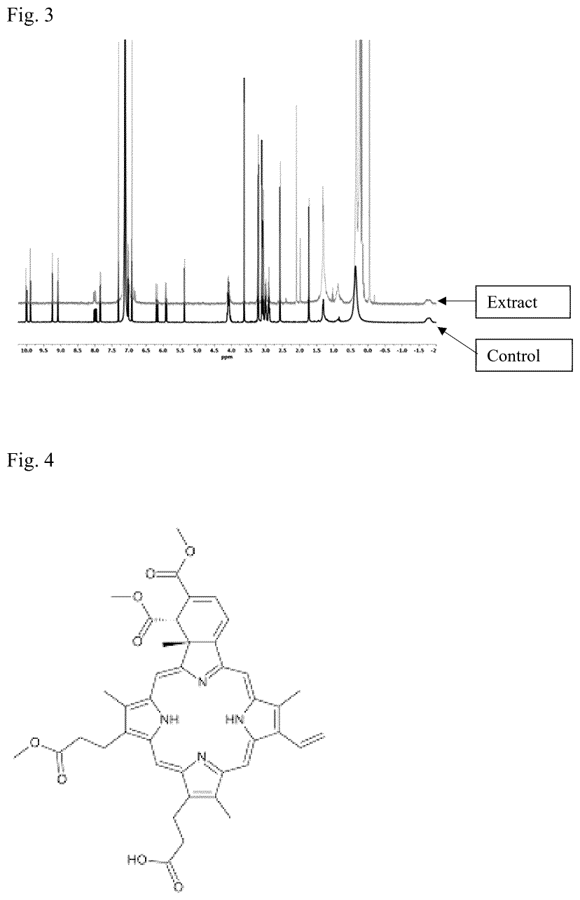

The solvent extract obtained in the Step 4 above was further characterized for the presence of verteporfin as follows. 5.22 grams of the deep green solution of Step 4 was placed in a weighing disk inside a fume hood overnight. Two sets of identical samples were prepared according to the above procedure. The first set of green residues was subjected to 1H NMR using deuterated benzene as solvent and the spectra is shown in FIG. 3. Pure verteporfin was also measured as the control sample. FIG. 3 shows 1H NMR of the green residue from solvent extraction of silicone film containing verteporfin vs control sample just containing verteporfin. The comparative plot in FIG. 3 demonstrates lower verteporfin concentration and several additional components (PDMS related, hydrocarbon, and unknowns) extracted from the silicone film that have affected the verteporfin spectral profile of extract somewhat, still it is clear that verteporfin is present in the extract. Further spiking of the extract solution with control verteporfin (not shown) confirmed that the verteporfin extracted from matrix was not chemically altered.

The weight of the residue from the second set of samples was measured, which is 0.0426 g. The control used had 0.504 grams of pure silicone film subjected to ethyl acetate extraction following the same procedure, with 0.0307 grams of residue was measured after the extraction solution dried in fume hood at ambient condition overnight. Based on the above it is calculated 0.0119 grams verteporfin was extracted from the 0.5 grams API-containing silicone film; from the difference of these two residue weight measurements (API containing and API-free silicone film). In view of the above, API verteporfin is readily leached out from the silicone carrier in ester type solvent.

### Example 3. Characterization of Adhesion Performance of Cured Silicone Films Containing Verteporfin

### Testing Samples Preparation.

As a Holding Strength testing specimen was used Synthetic Substrate, Polyester Film (0.05 Inch Thick Duralar® Film), Grafix Plastics, Maple Heights, Ohio. 1.5 inch wide PSA (pressure sensitive adhesive) coated polyester mesh (Lot #16204, Innovize, St Paul, Minn.) was placed along the cutting line to hold together the two half pieces of the Duralar film of 4 inches by 11 inches described above. Equal amount of the two-part silicone TSA composition prepared according to step 2 of Example 1 was mixed and applied onto the mesh to cover the entire area of the mesh using a conventional rubber spatula. The samples were cured for around 2 minutes at 31 deg C. and maintained at this temperature overnight. Five pieces of 1-inch-wide strips of each of the covered mesh samples were cut for testing.

As a Peel Test Sample was used Synthetic Substrate (Polyester Film, 0.05 Inch Thick Duralar® Film, Grafix Plastics, Maple Heights, Ohio). A 5 inch by 5 inch PSA (pressure sensitive adhesive) coated on polyester mesh (Lot #16204, Innovize, St Paul, Minn.) was placed on the polyester substrate of the same dimensions. The two-part silicone TSA composition prepared according to step 2 was mixed and applied onto the mesh evenly to cover the entire area of the mesh using a conventional rubber spatula. 5 pieces of 1-inch-wide specimens were cut for testing after each of the covered mesh samples were dried.

### Adhesion Testing.

Holding Strength Measurement: This test evaluated the force required to separate the substrate approximated with the PSA coated mesh and the applied silicone TSA composition. This method was based on ASTMF2458, a standard test method for wound closure strength in tissue adhesives and sealants. Synthetic substrate (Mylar) was used for the test. The width of the synthetic substrate was 1 inch and the strain rate was 20 inches/minute. 10.2 lb force had been measured which is the average of 5 specimens, which is the holding force for the polyester substrate. This force is comparable to the pure silicone two-part adhesive without API verteporfin.

### Peel Strength Measurement

The T-peel strength test was performed following ASTM F2256: Standard test method for Strength Properties of Tissue Adhesives in T-Peel by Tension Loading. The average peel strength of mesh coated with silicone-based TSA in T-peel configuration was performed at a strain rate of 10 inches/minute. 2.1 lb peel force had been measured which is the average of 5 specimens, which was also comparable to the pure silicone two-part adhesive without API verteporfin.

In view of the above, Adhesion, Holding Strength, Peel Strength are not significantly affected by the presence of verteporfin in the curable silicone composition.

### Example 4. Effect of Verteporfin on Crosslinking or Curing of the Silicone TSA

The following experiments were conducted to determine the limit of the loading of verteporfin in the instant silicone-based adhesive. For testing, 1, 3, 6 and 9% of verteporfin was incorporated into a silicone formulation as described below, similarly to the Example 1 as follows:

### Two-Part Medicant/API Containing Silicone Adhesive Preparation—1% Verteporfin Loading.

Part A: 9 grams of Elkem 55 experimental base (also known as Elkem Silbione 4020-55, containing vinyl terminated polydimethyl silicone base polymer and fumed silica particles) was mixed with 1 grams of the platinum catalyst master batch prepared according to step 1 of example 1, using a high-speed centrifugal mixer (FlackTek DAC150 FV-K) at 3470 rpm for 3 minutes.

Part B: 8.82 grams of Elkem 55 experimental base was mixed with 0.882 grams of polymethylhydro-co-polydimethyl siloxane cross linker (Gelest HMS H301), 0.294 grams of SiH terminated polydimethylsiloxane chain extender (Gelest DMS H21) and 0.204 grams of Verteporfin (Sigma Aldrich, SKU1711461) using a high-speed centrifugal mixer (Flack Tek DAC150 FV-K) at 3470 rpm for 3 minutes.

### Two-Part Medicant/API Containing Silicone Adhesive Preparation—3% Verteporfin Loading. (Similar to Described in Example 1).

Part A: 90 grams of Elkem 55 experimental base (also known as Elkem Silbione 4020-55, containing vinyl terminated polydimethyl silicone base polymer and fumed silica particles) was mixed with 10 grams of the platinum catalyst master batch prepared according to step 1 of example 1, using a high-speed centrifugal mixer (FlackTek DAC150 FV-K) at 3470 rpm for 3 minutes. This composition had viscosity of 32,090 CPs.

Part B: 84.6 grams of Elkem 55 experimental base was mixed with 8.46 grams of polymethylhydro-co-polydimethyl siloxane cross linker (Gelest HMS H301), 2.82 grams of SiH terminated polydimethylsiloxane chain extender (Gelest DMS H21) and 6.12 grams of verteporfin (Sigma Aldrich, SKU1711461) using a high-speed centrifugal mixer (Flack Tek DAC150 FV-K) at 3470 rpm for 3 minutes. This composition had a viscosity of 31,160 CPs.

### Two-Part Medicant/API Containing Silicone Adhesive Preparation—6% Verteporfin Loading.

Part A: 9 grams of Elkem 55 experimental base (also known as Elkem Silbione 4020-55, containing vinyl terminated polydimethyl silicone base polymer and fumed silica particles) was mixed with 1 grams of the platinum catalyst master batch prepared according to step 1 of example 1, using a high-speed centrifugal mixer (FlackTek DAC150 FV-K) at 3470 rpm for 3 minutes.

Part B: 7.92 grams of Elkem 55 experimental base was mixed with 0.792 grams of polymethylhydro-co-polydimethyl siloxane cross linker (Gelest HMS H301), 0.264 grams of SiH terminated polydimethylsiloxane chain extender (Gelest DMS H21) and 1.224 grams of verteporfin (Sigma Aldrich, SKU1711461) using a high-speed centrifugal mixer (Flack Tek DAC150 FV-K) at 3470 rpm for 3 minutes.

### Two-Part Medicant/API Containing Silicone Adhesive Preparation—9% Verteporfin Loading.

Part A: 9 grams of Elkem 55 experimental base (also known as Elkem Silbione 4020-55, containing vinyl terminated polydimethyl silicone base polymer and fumed silica particles) was mixed with 1 grams of the platinum catalyst master batch prepared according to step 1 of example 1, using a high-speed centrifugal mixer (FlackTek DAC150 FV-K) at 3470 rpm for 3 minutes.

Part B: 7.38 grams of Elkem 55 experimental base was mixed with 0.738 grams of polymethylhydro-co-polydimethyl siloxane cross linker (Gelest HMS H301), 0.246 grams of SiH terminated polydimethylsiloxane chain extender (Gelest DMS H21) and 1.836 grams of verteporfin (Sigma Aldrich, SKU1711461) using a high-speed centrifugal mixer (Flack Tek DAC150 FV-K) at 3470 rpm for 3 minutes.

The impact of the medicant on the catalytic activity of platinum catalyst was evaluated using a curing time measurement (ASTMC679). This test method is for tack-free time of elastomeric sealants, which consists of lightly touching a surface of a curing elastomer with a polyethylene film at regular intervals until the elastomer does not attach itself to the film and film appears clean when peeled from the surface. This indicates the silicone elastomer is fully cross linked or cured. Table 1 presents the results of measuring the tack-free times in seconds obtained in this test. Loading of 0% corresponds to silicone TSA without any verteporfin.

TABLE 1
Tack-free times vs. concentration of Verteporfin in silicone TSA

| Verteporfin Loading in silicone | |
|---|---|
| TSA (% by Wt.) | Tack -free Time (s) |
| 0 | 115 |
| 1 | 115 |
| 3 | 125 |
| 6 | 140 |
| 9 | 1020 |

The above experimental results provide for means to determine the limit of the loading of verteporfin in the instant silicone-based adhesive. For testing, 1%, 3%, 6% and 9% of verteporfin was incorporated into a silicone formulation. This study shows 3% and lower loading of verteporfin has minimum impact on catalytic activity. Up to 6% of loading of verteporfin still can result in potentially workable topical skin adhesive formulation, but 9% and more verteporfin results in substantial degradation of the catalytic activity and substantially delayed cross-linking/curing.

### Example 5. Verteporfin Release into Bovine Plasma Characterization with UV-Vis Spectroscopy

Analytical data on quantification of verteporfin in solution via UV-Vis absorption is presented. Different solutions with known concentration of verteporfin were made in Isopropyl Alcohol (IPA). Subsequently their UV-Vis absorption spectra were obtained. For all the solution a peak at 690 nm was observed. In the scientific literature, there are several reports on identification and quantification of verteporfin at this specific wavelength: Calori, Italo Rodrigo, et al. “Self-aggregation of the proteolytic forms of Verteporfin: An in silico and in vitro study.” Journal of Molecular Liquids 352 (2022): 118640. Pellosi D S, et al., Pluronic® P123/F127 mixed micelles delivering sorafenib and its combination with verteporfin in cancer cells. Int J Nanomedicine. 2016 Sep. 6; 11:4479-4494. Simionescu, S., et al., (2017). Polymeric membrane for verteporfin purification. Materiale Plastice, 54(1), 14.

Characterization of release of verteporfin into Bovine plasma from cured silicone TSA loaded with verteporfin (3% w/w) was performed. The results of this in-vitro experiment are demonstrating the release of verteporfin in a biological fluid and quantifying the amount of verteporfin released after 4 days, corresponding to the middle of the wound closure inflammatory phase.

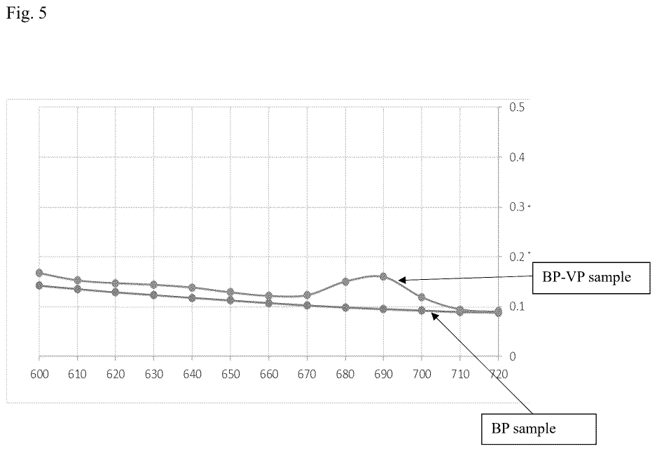

The cured silicone film was exposed to freshly separated Bovine plasma for 4 days. A distinct peak appears at 690 nm in UV-Vis absorption graph. This peak has been associated with verteporfin in the literature. Bovine plasma was obtained from Bovine blood by means of centrifuging (1500 G, 20 minutes). A 0.0534 g piece of cured silicone film (13×9×0.3 mm) loaded with verteporfin was placed in a glass vial with 10 ml of Bovine plasma sample. Another vial filled only with bovine plasma sample was prepared as blank sample. The samples were incubated in the oven at approximately 35° C. (temperature fluctuated between 33-38° C.) for 4 days. Thereafter the samples were scanned for UV-Vis absorption from 200-800 nm (rate: 10 nm/s). The measurement was executed using 1 ml (0.5×2 cm) Quartz cuvettes. It has been reported in the literature that verteporfin has an indicative absorption peak at 690 nm. In this study a distinct peak at 690 is also detectable which is not present in the blank sample. FIG. 5 shows UV-Vis absorption measurements for blank bovine plasma sample (BP sample) and bovine plasma exposed to silicone containing verteporfin (BP-verteporfin sample), having a characteristic peak at around 690 nm. The data indicates release of the verteporfin into bovine plasma from cured silicone film.

### Example 6. Verteporfin Quantification with UV-Vis Spectroscopy

USP reference sample of verteporfin was used for preparation of known concentration solution in isopropyl alcohol (IPA). A total number of five dilutions were made for this study. A 118 micromolar verteporfin solution was made as the main dilution solution. All the other four dilutions were made by further dilution of this solution. Verteporfin is readily soluble in IPA, the solutions were manually shaken until verteporfin powder disappeared and solution turned to a homogenous green color. Table 2 presents sources of verteporfin and IPA

TABLE 2
Sources of Verteporfin, IPA

| Chemical name | Lot | Supplier |
|---|---|---|
| Verteporfin | R049D0 | USP |
| Isopropyl Alcohol (95%) | C22K0501 | VWR |

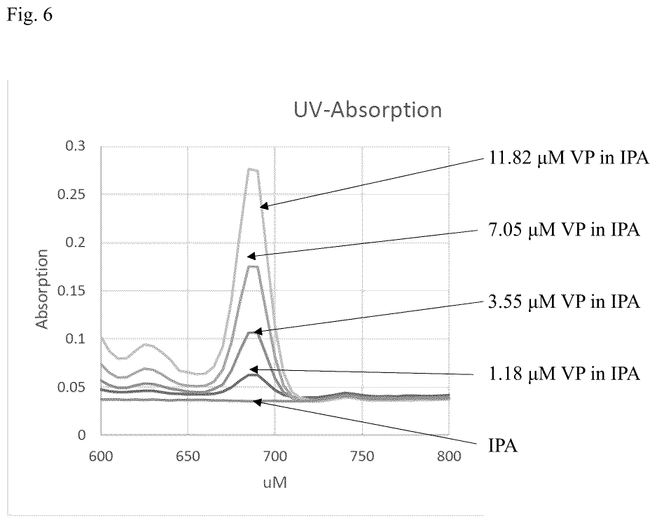

Solutions were scanned for UV-Vis absorption (SpectraMAX-M3) from 600-800 nm (rate: 5 nm/s). The measurements were executed using 3.5 ml (pass length 10 mm) Quartz cuvettes. As expected, a peak appeared at 690 nm. An increase in the intensity of the peak was observed with increasing the concentration of verteporfin. FIG. 6 presents the absorption spectra of five different verteporfin dilutions (verteporfin-IPA) and IPA blank. Table 3 shows the maximum absorption at 690 nm for each solution.

TABLE 3
Maximum absorption at 690 nm for each solution

| Verteporfin P-IPA Solution | |
|---|---|
| Concentration (micro-moles) | UV-Vis absorption |
| 11.82 | 0.2744 |
| 7.05 | 0.1751 |
| 3.55 | 0.1063 |
| 1.18 | 0.0623 |

According to the Beer-Lambert law, there is a linear relationship between the absorbance (A) and the concentration (c), molar absorption coefficient (F) and optical path length of a solution (1): A=εcl

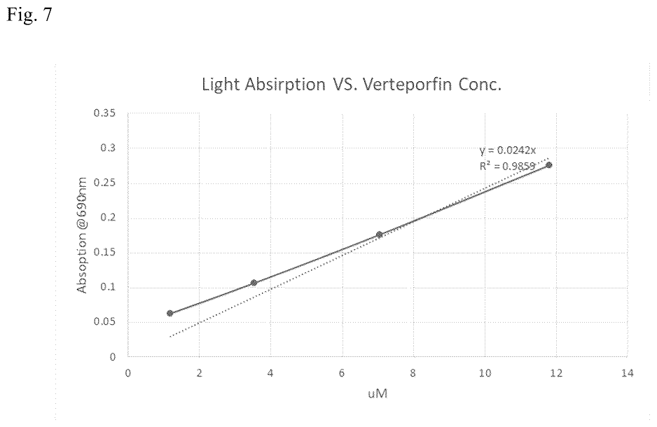

FIG. 7 presents a calibration curve obtained based on Beer-Lambert law from the data of Table 3, measured at λ max, i.e. at 690 nm. The obtained F value for verteporfin in IPA is: 0.0242 microM-1cm-1

Based on the calibration curve of FIG. 7, the absorption recorded for silicone TSA films with Verteporfin released into bovine plasma corresponds to 6.59 microM in 10 ml bovine plasma: Absorption at 690 nm: 0.1595. Released concentration based on Calibration Curve (microM): 6.59.

Given that the molecular weight of verteporfin is 718.79 g/mol, the actual mass of verteporfin released from a 0.0534 grams silicone film loaded with 3% verteporfin in 10 ml of plasma is 47.37 micrograms.

### Example 7. Verteporfin Release into Bovine Plasma for Various Verteporfin Loadings in Silicone TSA Films

The testing was performed using methodology similar to Examples 5, 6, and utilizing the obtained F value for verteporfin of 0.0242 microM-1 cm-1 derived above.

The released amount of verteporfin in bovine plasma was measured at different time points. Two loading concentrations (3% and 6% w/w) of verteporfin in silicone TSA film were tested.

A release profile was thereby obtained for silicone TSA film loaded with verteporfin in bovine plasma. verteporfin was dispersed in part B (according to the procedure disclosed above for 3% and 6% verteporfin loading preparations, Examples 1, 4 above. The silicon elastomer film was obtained by mixing part A and Part B using static mixer. The obtained cured films had the approximate thickness of 0.5 mm.

Table 4 presents the sources of materials

TABLE 4
Materials used

| Material | Lot |
|---|---|
| Silicone TSA | A: 2023136-01 and B: 2023136-02 |
| USP Reference Verteporfin | R049D0 |
| Bovine Whole Blood in | 23L52531 |
| Na-Citrate, Donor Lampire) | |

The silicone TSA samples in bovine plasma were placed in an oven at 37° C. (±3° C.) and tested at time intervals of 6 hours, 24 hours, 48 hours and 1 week for the released verteporfin. 20 ml glass scintillation vials were used as test containers.

Table 5 presents details of cumulative verteporfin release samples

TABLE 5
Cumulative verteporfin release test samples

| | | | Approximate total | |
|---|---|---|---|---|
| | Number | Film weight | Verteporfin weight in | |
| Sample name | of Vials | (grams) | the film (micrograms) | Bovine Plasma (grams) |
| Blank | 1 | N/A | N/A | 5 g- to be tested at 6 hours, |
| | | | | 24 hours, 48 hours and 1 |
| | | | | week |
| Silicone TSA film | 1 | 0.0259 | 777 | 5 g-to be tested at 6 hours, |
| with 3% Verteporfin | | | | 24 hours, 48 hours and 1 |
| loading | | | | week |
| Silicone TSA film | 1 | 0.0268 | 1608 | 5 g-to be tested at 6 hours, |
| with 6% Verteporfin | | | | 24 hours, 48 hours and 1 |
| loading | | | | week |

Bovine Plasma density averages around 1.1 g/ml. The volume of testing vials is estimated at 4.5 ml. The film dimensions were kept approximately similar to the previous example (96 hours) at 1.2 cm², the thickness of the film was between 0.1-0.3 mm. The film density was lower than bovine plasma and the film sat on the surface of the liquid for the entire length of the study. For this reason, it is assumed that verteporfin was released from one side of the film.

The pulled samples were scanned for UV-Vis absorption (SpectraMAX-M3) from 600-800 nm (rate: 5 nm/s) at intervals indicated in Table 5. The measurements were executed using 3.5 ml (pass length 10 mm) Quartz cuvettes. As expected, a peak appeared at 690 nm. An increase in the intensity of the peak was observed with increasing the release time.

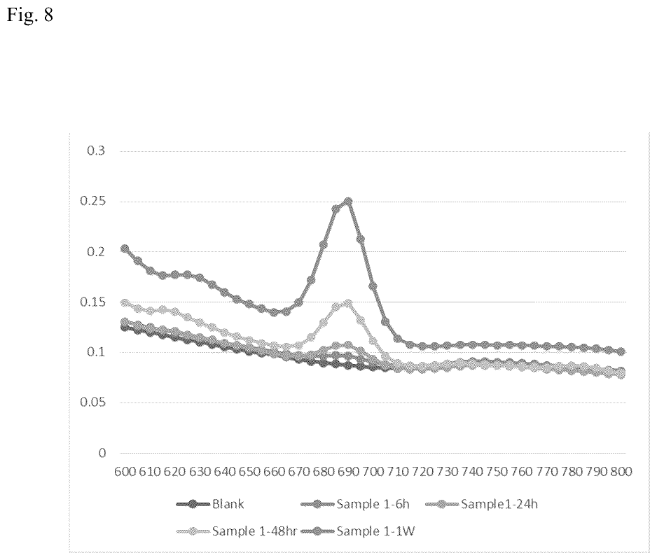

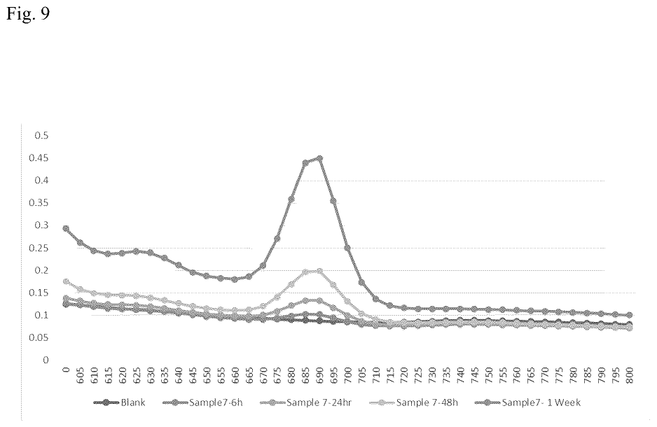

FIGS. 8 and 9 show the UV-Vis absorption spectra of Bovine Plasma after incubation with silicone TSA films having 3% verteporfin and 6% verteporfin, after 6 hours, 24 hours, 48 hours and one week incubation.

FIG. 8 shows the results for 3% verteporfin loaded silicone TSA film, with the lowest curve corresponding to blank sample (no verteporfin loading), next higher curve corresponding to 6 hours incubation, next higher curve corresponding to 24 hours incubation, next higher curve corresponding to 48 hours incubation, and the highest curve corresponding to 1 week incubation.

FIG. 9 shows the results for 6% verteporfin loaded silicone TSA film, with the lowest curve corresponding to blank sample (no verteporfin loading), next higher curve corresponding to 6 hours incubation, next higher curve corresponding to 24 hours incubation, next higher curve corresponding to 48 hours incubation, and the highest curve corresponding to 1 week incubation.

The results clearly indicate significant release of verteporfin over time, with higher incubation time resulting in more verteporfin release, and higher loading of verteporfin in the TSA film resulting in higher amount of verteporfin released from TSA film.

To calculate the concentration of verteporfin in bovine plasma solution, the blank solution absorption at 690 nm (0.087) was subtracted from the measured absorption for each timepoint. Tables 6 and 7 present the absorption values and corresponding verteporfin mass cumulatively released from 3% and 6% verteporfin containing silicone TSA films in Bovine Plasma over time.

TABLE 6
Absorption values and corresponding verteporfin mass release for
3% verteporfin containing silicone TSA films in Bovine Plasma.

| | | | | Estimated | Released |
|---|---|---|---|---|---|
| | | | | Released Mass of | Verteporfin |
| | | | Adjusted | Verteporfin / | Mass, |
| Release | | UV-Vis | Absorption | micrograms per 1 | micrograms |
| Period | | absorption@690 nm | @690 nm | Liter | into 4.5 mL |
| 6 | hours | 0.096 | 0.009 | 267.3 | 1.202 |
| 1 | day | 0.1072 | 0.0202 | 559.9 | 2.697 |
| 2 | days | 0.1487 | 0.0617 | 1832.6 | 8.238 |
| 7 | days | 0.2501 | 0.1631 | 4844.4 | 21.776 |

TABLE 7
Absorption values and corresponding verteporfin mass release for
6% verteporfin containing silicone TSA films in Bovine Plasma.

| | | | | | Released |
|---|---|---|---|---|---|
| | | | | Released Mass of | Verteporfin |
| | | | Adjusted | Verteporfin / | Mass, |
| Release | | UV-Vis | Absorption | micrograms per 1 | micrograms |
| Period | | absorption@690 nm | @690 nm | Liter | into 4.5 mL |
| 6 | hours | 0.1013 | 0.0143 | 424.7 | 1.911 |
| 1 | day | 0.1323 | 0.0453 | 1345.5 | 6.055 |
| 2 | days | 0.1986 | 0.1116 | 3314.7 | 14.916 |
| 7 | days | 0.4498 | 0.3628 | 10775.9 | 48.491 |

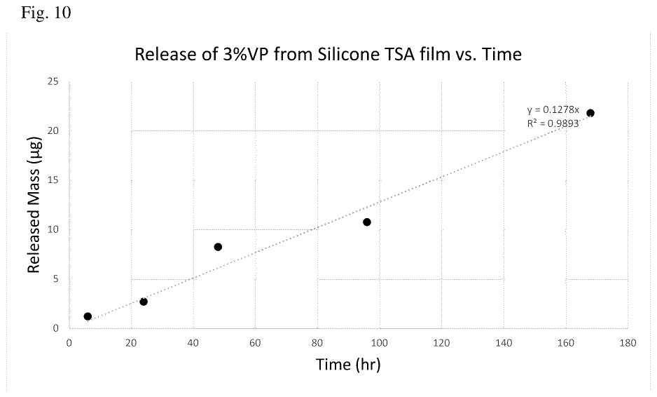

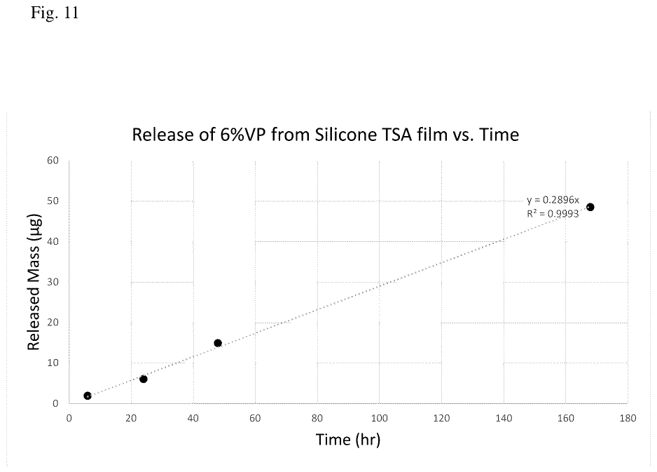

The data presented in Tables 6, 7 is also presented in FIGS. 10, 11 as a plot of cumulative mass release vs. time. FIG. 10 shows cumulative verteporfin mass release vs. time for 3% verteporfin containing silicone TSA films in Bovine Plasma. Release mass for 96 hours data point was normalized to be comparable to other points in terms of film mass and plasma volume.

FIG. 11 shows cumulative verteporfin mass release vs. time for 6% verteporfin containing silicone TSA films in Bovine Plasma.

The obtained data suggests a substantially linear release profile of verteporfin form verteporfin-containing silicone TSA films into bovine plasma. Even after 6 hours presence of verteporfin in bovine plasma is detectable and quantifiable. The release is faster in higher loaded film (6%) compared to the lower loading concentration (3%), with controllable sustained release of verteporfin over the course of the entire inflammatory phase (7 days).

Based on the above results, around 22 μg of verteporfin was released from 3% verteporfin loaded sample into bovine plasma after 7 days, and 48 μg of verteporfin released from 6% verteporfin loaded sample into bovine plasma after 7 days, the surface areas of silicone patch in both cases were around 1.2 cm². The embodiments of the present invention provide for a high-level dose of verteporfin incorporated into silicone-based TSA and released from the silicone film matrix.

In embodiments, a method of treating a wound comprising the steps: Controlled release of verteporfin over inflammatory phase directly onto the target fibroblast cells, blocking the scar formation pathway leading to lower scarring and/or scarless wound recovery.

In embodiments, verteporfin can be incorporated into one or both syringes containing the curable silicone TSA components (part A and/or Part B). Verteporfin can also be mixed into the Part A and/or Part B immediately prior to application of silicone TSA. In an alternative embodiment, verteporfin van be contained in a solution in a third syringe and co-expressed with Part A and Part B thorough a static mixer for incorporation into the resulting silicone TSA film, using a two-barrel syringe with a static mixer.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

I/We claim:

1. A topical liquid silicone skin adhesive formulation having two co-reactive parts comprising:
Co-reactive Part A comprising
(A1) at least one vinyl terminated polydimethyl silicone base polymer in liquid form,
(A2) fumed silica solid particles in silicone base polymer A1, and
(A3) a platinum catalyst capable of catalyzing the reaction of silicone base polymer at 20-40° C.; and
Co-reactive Part B comprising
(B1) at least one vinyl terminated polydimethyl silicone base polymer in liquid form,
(B2) fumed silica particles in silicone base polymer B1,
(B3) a cross-linker to increase crosslink density upon reaction,
(B4) a chain extender to effectively increase the molecular weight of the base silicone polymer by reaction, and
(B5) verteporfin in solution.

2. The topical skin adhesive of claim 1, wherein the at least one vinyl terminated polydimethyl silicone base polymer has a number average molecular weight in the range of 10000-200000.

3. The topical skin adhesive of claim 1, wherein the at least one viny terminated polydimethyl silicone base polymer is a blend of a low viscosity vinyl terminated polydimethyl silicone based polymer and a higher viscosity vinyl terminated polydimethyl silicone based polymer, wherein the number average molecular weight of the low viscosity vinyl terminated polydimethylsiloxane polymer ranges from 1000 to 10000 and the higher viscosity vinyl terminated polydimethylsiloxane polymer has a number average molecular weight of 25000 to 150000.

4. The topical skin adhesive of claim 1, wherein the vinyl terminated polydimethyl silicone base polymer B1 has a number average molecular weight in the range of 25000 to 150000.

5. The topical skin adhesive of claim 1, wherein the platinum catalyst comprises a platinum tetramethyldivinyl disiloxane diethyl maleate complex, the cross-linker comprises a polymethylhydro-co-polydimethyl siloxane cross linker, the chain extender comprises a SiH terminated polydimethylsiloxane chain extender.

6. The topical skin adhesive of claim 2, wherein the verteporfin concentration is from 1 to 9% by weight relative to Parts A and B.

7. The topical skin adhesive of claim 3, wherein the verteporfin concentration is from 3 to 6% by weight to Parts A and B.

8. The topical skin adhesive of claim 3, wherein said Part A and Part B are storage stable.

9. The topical skin adhesive of claim 3, wherein the formulation after mixing is tack-free cured in under 3 minutes.

10. The topical skin adhesive of claim 3, wherein the formulation is tack-free cured in under 3 minutes after mixing when applied to a skin of a mammal and forms a solid film dressing, wherein said dressing exhibits a sustained release of verteporfin.

11. The topical skin adhesive of claim 9, wherein said dressing exhibits a sustained release of verteporfin over at least 24 hours after application.

12. The topical skin adhesive of claim 9, wherein said dressing exhibits a sustained release of verteporfin over at least 7 days after application.

13. The topical skin adhesive of claim 1, further comprising an anti-infective agent that comprises triclosan or chlorhexidine gluconate or a silver compound, or combinations thereof.

14. The topical skin adhesive of claim 13, wherein said anti-infective agent is mixed into Part A or Part B.

15. A system for applying the topical skin adhesive of claim 1, comprising:
a) A dual barrel syringe containing Part A in a first syringe and Part B in a second syringe,
wherein the dual barrel syringe terminates in a static mixer that is configured for mixing and co-expressing Part A and Part B.

16. A kit comprising the system of claim 15 and a flexible porous mesh configured for application onto a tissue and for approximating edges of a wound, said mesh having a pressure sensitive adhesive disposed on one side thereof.

17. The kit of claim 16, wherein an anti-infective agent that comprises triclosan or chlorhexidine gluconate or a silver compound, or combinations thereof, is disposed on said mesh.

18. A method of decreasing wound scars or wound scar formation by the application of a liquid curable silicone formulation according to claim 1 on a tissue surface.
